(19) United States
(12) Patent Application Publication
(10) Pub. No.: US 2009/0081194 A1
(43) Pub. Date: Mar. 26, 2009

(54) COMPOSITIONS AND METHODS FOR REDUCING RISK OF DEVELOPMENT, OR SEVERITY, OF INAPPROPRIATE IMMUNE RESPONSE IN EYES

(76) Inventors: Carolyn M. Kalsow, Mendon, NY (US); Brian Levy, Rochester, NY (US); Timothy L. Comstock, Rochester, NY (US); William T. Reindel, Webster, NY (US)

Correspondence Address: Bausch & Lomb Incorporated, One Bausch & Lomb Place, Rochester, NY, 14604-2701, US

(21) Appl. No.: 12/062,585

(22) Filed: Apr. 4, 2008

Related U.S. Application Data
(60) Provisional application No. 60/910,006, filed on Apr. 4, 2007.

(51) Int. Cl. A61K 38/00; A61K 39/395; A61K 31/7088; A61K 31/47; A61K 31/473; A61K 31/59; A61K 31/592; A61K 31/593; A61P 37/00; A61P 27/02

(52) U.S. Cl. 424/130.1; 514/2; 514/44; 514/313; 514/297; 514/167

(57) ABSTRACT

A composition for reducing the risk of development, or severity, of an inappropriate immune response in an eye comprises an antagonist to at least a human TLR, an antagonist to at least a coreceptor of human TLR, a compound that is capable of inhibiting an activation of a human TLR signaling pathway, or a combination thereof. The composition can be used to reduce the risk of development, or severity, of contact lens-associated corneal infiltrates. The composition can be formulated into an eye drop or a contact lens-treating, -storing, -cleaning, -disinfecting, or -wetting solution.

### CROSS REFERENCE

This application claims the benefit of Provisional Patent Application No. 60/910,006 filed Apr. 4, 2007 and is incorporated herein by reference.

### BACKGROUND OF THE INVENTION

The present invention relates to compositions and methods for reducing risk of development, or severity, of inappropriate immune response in the eyes. In particular, the present invention relates to compositions and methods for reducing risk of development, or severity, of contact lens-associated corneal infiltrates.

The term “corneal infiltrates” refers to inflammatory cells of the immune system that enter the cornea in response to stressors such as toxins, ocular irritants, or other materials foreign to the ocular environment. Corneal infiltrates represent a vexing ocular condition because they may present benignly as a few clusters of inflammatory cells in the cornea of both asymptomatic contact lens-wearing patients and patients who do not wear contact lenses. These infiltrates may vary in appearance and severity from asymptomatic collections of cells to painful, red eyes with a breach of epithelial integrity. Corneal infiltrates are typically composed of polymorphonuclear leukocytes (neutrophils), but may also contain lymphocytes and macrophages. Infiltrating cells may migrate from the limbal vasculature or from the tear film in response to local tissue damage and chemotactic factors, induced by antigens and toxins from the environment including components from microbial organisms. Contact lens-associated corneal infiltrates (“CLACIs”) occur in a small percentage but notable number of contact lens wearers, and are most often culture negative. Any one or a combination of multiple mechanical, hypoxic, toxic, or irritating stimuli associated with contact lens use can induce proinflammatory responses that lead to infiltration of inflammatory cells into the cornea. In one aspect, corneal infiltrates may be associated with the presence of microbes at the ocular surface. These microbes may not directly cause tissue damage (infection) but can elicit an innate immune response by release of cellular components such as endotoxin, cell wall materials, or nucleic acids. M. W. Robboy et al., Eye & Contact Lens, Vol. 29, No. 3, 146 (2003).

The normal flora of a healthy eye includes several types of microorganisms such as Corynebacterium xerosis, Staphylococcus epidermis, saprophytic fungi, Neisseria species, Moraxella species, and nonhemolytic Streptococci. Upon death and disintegration as well as part of the normal growth process, these microorganisms release chemicals and cellular products, which are foreign to the host and activate resident ocular surface cells to produce cytokines and chemokines that can induce a congregation of inflammatory cells of the innate immune system, manifesting as corneal infiltration by these cells.

Host defense against challenge by foreign materials is elicited by the immune system, which consists of innate immunity and acquired (adaptive) immunity. Adaptive immunity is mediated by T and B lymphocytes that proliferate clonally in response to a specific pathogen or antigen. The generation of acquired immune responses requires a number of days after the host is exposed to the challenge. In contrast, the innate immune system is activated soon after such pathogenic or antigenic challenge to provide nonspecific protection before the acquired immunity system becomes fully effective.

It was recently discovered that the rapid innate immune response is due in part to a family of cellular receptors termed “Toll-like receptors” (“TLRs”) that have evolved to recognize some common structural features of the diverse microorganisms, which features are referred to as “pathogen-associated molecular patterns” (or “PAMPs”). To date, at least ten mammalian TLRs have been identified, and ligands that activate some of these TLRs have been ascertained. K. Takeda et al., Annual Rev. Immunol., Vol. 21, 335 (2003). For example, TLR1 recognizes tri-acyl lipopeptides of bacteria and Mycobacteria. TLR2 recognizes lipoproteins and lipopeptides of a variety of Gram-negative bacteria, peptidoglycan and lipoteicholic acid of Gram-positive bacteria, lipoarabinomannan of Mycobacteria, and several types of atypical lipopolysaccharides (“LPSs”) of Leptospira interrogans and Porphyromonas gingivalis. TLR3 recognizes double-stranded RNA (“dsRNA”) of viruses. TLR4 recognizes LPSs, which are outer-membrane components of Gram-negative bacteria and are structurally different from the atypical LPSs recognized by TLR2. TLR5 recognizes flagellin of Gram-negative bacteria. TLR6 recognizes di-acyl lipopeptides. Id. Human TLR7 and TLR8 recognize imidazoquinoline compounds, which are structurally related to guanosine nucleoside. Thus, they are predicted to recognize nucleic acid-like structure of viruses or bacteria. K. Takeda et al., Int. Immunol., Vol. 17, No. 1, 1 (2005). In fact, TLR8 recently has been indicated to recognize single-stranded RNA of viruses (“ssRNA”). TLR9 recognizes the unmethylated CpG motifs of bacterial DNA. To date, ligands of TLR10 have not been ascertained. Additional TLRs may be discovered in the future as knowledge of the immune system continues to expand. TLR expression and function have been demonstrated in the eye. J. H. Chang et al., Br. J. Opthalmol., Vol. 90, 103 (2006).

It has been shown that some TLRs act in concert with other TLRs or coreceptors (such as CD14 or MD-2) to initiate intracellular inflammatory cascades, which have the ultimate goal of elimination of the foreign materials from the body. Among the most prominent and best characterized of these cascades is that leading to the activation of the transcription factor NF-κB, which, in turn, activates the genes for production of many proinflammatory factors (such as TNF-α, IL-1, and IL-12). In addition, TLRs can also initiate mitogen-activated protein kinase (“MAPK”) signaling cascades and thus activate other transcription factors, including activator protein 1 (“AP-1”) and Elk-1. G. Zhang et al., J. Clin. Invest., Vol. 107, No. 1, 13 (2001).

Therefore, components of microbial cells of the normal ocular flora that are not quickly carried away from the cornea surface, for example by insufficient production of tear or by being trapped under a contact lens, coupled with some minor breach of the cornea epithelial layer, can elicit an innate immune response in healthy contact lens wearers. Such an immune response, in turn, can promote CLACIs. These corneal infiltrates further synthesize and release proinflammatory cytokines such as IL-1β, IL-3, IL-5, IL-6, IL-8, TNF-α (tumor necrosis factor-α), GM-CSF (granulocyte-macrophage colony-stimulating factor), and MCP-1 (monocyte chemotactic protein-1). These released cytokines then further attract more immune cells to the affected site, amplifying the response of the immune system to defend the host against the foreign pathogen. For example, IL-8 and MCP-1 are potent chemoattractants for, and activators of, neutrophils and monocytes, respectively, while GM-CSF prolongs the survival of these cells and increases their response to other proinflammatory agonists. TNF-α can activate both types of cell and can stimulate further release of IL-8 and MCP-1 from them. IL-1 and TNF-α are potent chemoattractants for T and B lymphocytes, which are activated to produce antibodies against the foreign pathogen.

Although an inflammatory response is essential to clear foreign materials from the site of invasion, a prolonged or overactive inflammatory response can be damaging to the surrounding tissues. For example, inflammation causes the blood vessels at the infected site to dilate to increase blood flow to the site. As a result, these dilated vessels become leaky. After prolonged inflammation, the leaky vessels can produce serious edema in, and impair the proper functioning of, the surrounding tissues (see; e.g., V. W. M. van Hinsbergh, Arteriosclerosis, Thrombosis, and Vascular Biology, Vol. 17, 1018 (1997)). In addition, a continued dominating presence of macrophages at the site of invasion continues the production of toxins (such as reactive oxygen species) and matrix-degrading enzymes (such as matrix metalloproteinases) by these cells, which are injurious to both the pathogen and the host's tissues. Therefore, an inappropriately vigorous activation of the immune system in response to non-infectious foreign materials should be controlled to limit the unintended damages to an otherwise healthy tissue.

In view of the delicate structure of the eye, there is a need to provide compositions and methods for reducing the risk of development, or severity, of inappropriate immune responses therein. In particular, it is also very desirable to provide compositions and methods for reducing the risk of development, or severity, of contact lens-associated corneal infiltrates.

### SUMMARY OF THE INVENTION

In general, the present invention provides compositions and methods for reducing the risk of development, or severity, of inappropriate immune response in an eye.

In one aspect, the present invention provides compositions and methods for reducing the risk of development, or severity, of CLACIs.

In another aspect, a composition of the present invention comprises an antagonist to at least one human TLR or a coreceptor of a human TLR.

In still another aspect, such an antagonist to at least one human TLR or a coreceptor of a human TLR is capable of down regulating a TLR signaling pathway.

In yet another aspect, a composition of the present invention comprises a compound that is capable of inhibiting an activation of a human TLR signaling pathway.

In a further aspect, a composition of the present invention is a contact-lens storing, cleaning, wetting, rinsing, or disinfecting solution and comprises an antagonist to at least one human TLR, an antagonist to a coreceptor of a human TLR, a compound that is capable of inhibiting an activation of a human TLR signaling pathway, or a combination thereof.

In yet another aspect, the present invention provides a method for reducing the risk of development, or severity, of an inappropriate immune response in an eye. The method comprises applying a composition to the eye, wherein the composition comprises an antagonist to at least one human TLR, an antagonist to a coreceptor of a human TLR, or a compound that is capable of inhibiting an activation of a human TLR signaling pathway, or a combination thereof.

In still another aspect, the present invention provides a method for reducing the risk of development, or severity, of CLACIs in a contact-lens wearer. The method comprises providing an antagonist to at least one human TLR, an antagonist to a coreceptor of a human TLR, or a compound that is capable of inhibiting an activation of a human TLR signaling pathway, to a composition that is used to store, clean, or disinfect a contact lens to be worn by such wearer.

Other features and advantages of the present invention will become apparent from the following detailed description and claims.

### BRIEF DESCRIPTION OF THE DRAWINGS

FIG. 1 shows ODN 2088 inhibition of neutrophil MIP-2 response.

FIG. 2 shows ODN 2088 inhibition of neutrophil KC response.

FIG. 3 shows ODN 2088 inhibition of neutrophil TNF-α response.

FIG. 4 ODN 2088 inhibition of neutrophil IL-6 response.

FIG. 5 shows the effect of the inhibitory ODN 2088 on neutrophil infiltrate after a compromised mouse cornea has been exposed to stimulatory ODN 1826, bacterial DNA, Pam3Cys, or LPS.

FIG. 6 shows ODN 2088 inhibition of corneal MIP-2, KC, and IP-10 response.

FIG. 7 shows the effect of the inhibitory ODN (having sequence TTAGGG) on the TLR activation of human cell lines by Pam3Cys, flagellin, or CpGB.

### DETAILED DESCRIPTION OF THE INVENTION

In general, the present invention provides compositions and methods for reducing the risk of development, or severity, of inappropriate immune response in an eye. As used herein, the term “inappropriate immune response” means a response of the body's immune system to a foreign material, such response being at an unwanted high level that results in a pathological condition.

In one aspect, the present invention provides compositions and methods for reducing the risk of development, or severity, of CLACIs.

In another aspect, such CLACIs are sterile infiltrates.

In still another aspect, a composition of the present invention comprises an antagonist to at least one human TLR or to a coreceptor of a human TLR. As used herein, the term “antagonists” to a TLR or to a coreceptor of TLR also includes compounds that inhibit or impede the expression of such receptor or coreceptors. In one embodiment, such antagonist is present in the composition at concentrations such that the composition is capable of reducing the risk of development, or the severity, of an inappropriate immune response in the eye.

In still another aspect, such an antagonist inhibits the binding of ligands to such TLR or coreceptor, which ligands are capable of activating such TLR or coreceptor, or the binding of such coreceptor to such TLR.

In yet another aspect, said at least one human TLR is selected from the group consisting of TLR1, TLR2, TLR3, TLR4, TLR5, TLR6, TLR7, TLR8, TLR9, TLR10, and combinations thereof.

In a further aspect, said coreceptor of a human TLR is selected from the group consisting of CD14, MD-2, and a combination thereof. CD-14 has been shown to be an essential coreceptor for TLR2 and TLR4 activation due to the required formation of the receptor complex comprising CD14 and TLR2 or TLR4 before the signaling cascades involving these TLRs are initiated. G. Zhang et al., J. Clin. Invest., Vol. 107, No. 1, 113 (2001); R. Arroyo-Espliguero et al., Heart, Vol. 90, 983 (2004). Growing evidence has suggested that an association of MD-2, a lipid binding protein, with the leucine-rich repeats (“LRRs”) of the extracellular domain of TLR4 is necessary for the initiation of the signaling cascade involving this TLR by LPS components of bacteria. See; e.g., T. L. Gioannini et al., PNAS, Vol. 101, No. 2, 4186 (2004).

In one aspect, a composition of the present invention comprises an anti-human antibody of TLR1, TLR2, TLR3, TLR4, TLR5, TLR6, TLR7, TLR8, TLR9, TLR10, CD14, MD-2, or combinations thereof. Many of these antibodies are available from eBioscience, San Diego, Calif. In one embodiment, such an antagonist is a monoclonal antibody. In another embodiment, such an antagonist is a recombinant antibody of TLR1, TLR2, TLR3, TLR4, TLR5, TLR6, TLR7, TLR8, TLR9, TLR10, CD14, MD-2, or combinations thereof.

In another aspect, a composition of the present invention comprises a soluble form of an extracellular domain of a TLR (“sTLR”) that recognizes a microbe-expressed molecular structure (“MEMS”). By binding to an MEMS, a sTLR renders it unavailable for binding to the corresponding TLR and activating the signaling cascade involving the same. Soluble TLRs are available from, for example, eBioscience, San Diego, Calif. These molecules may be cleaved into smaller fragments, for example, using enzymatic digestion, and those fragments that recognize a particular MEM at high affinity may be identified through binding assays that are well known in the art.

In still another aspect, a composition of the present invention comprises a soluble form of a CD14-binding extracellular domain of TLR4 (“sTLR4”), a soluble form of CD14 molecule (“sCD14”), or a soluble form of MD-2 (“sMD-2”). Such sTLR4 binds to CD14 and prevents it from binding to membrane-bound TLR4 and assisting in activating the signaling cascade involving the same. On the other hand, sCD14 and sMD-2 bind to LPS components of bacteria and prevent its binding to TLR4 and subsequent activation of this TLR. Soluble forms of extracellular domain of TLR4 and MD-2 have been shown to be effective in inhibiting LPS-elicited IL-8 release from U937 cells and NF-κB activation. H. Mitsuzawa et al., J. Immunol., Vol. 177, 8133 (2006). Soluble CD14 and MD-2 are available from, for example, IMGENEX, Corp., San Diego, Calif.

In another aspect, a composition of the present invention comprises a TLR-inhibiting oligodeoxynucleoside (“ODN”) that comprises at least three consecutive guanosine deoxynucleotides. In one embodiment, a composition of the present invention comprises a TLR-inhibiting ODN that comprises at least a GGG (“G-triplet”) or GGGG (“G-tetrad”) motif. In another embodiment, a composition of the present invention comprises a TLR-inhibiting single-stranded ODN that comprises multiple TTAGGG motifs (SEQ. NO. 1) or a sequence of TCCTGGCGGGGAAGT (SEQ. NO. 2). SEQ. NO. 1 is ubiquitously found in human telomeres. SEQ. NO. 2 is a synthetic ODN, known as ODN 2088, available from InvivoGen, San Diego, Calif. These ODNs have been shown to block the colocalization of CpG DNA, which is ubiquitously found in bacterial products, with TLR9 within endosomal vesicles. I. Gursel et al., J. Immunol., Vol. 171, 1393 (2003); L. L. Stunz et al., Eur. J. Immunol., Vol. 171, No. 3, 1212 (2002). Preferably, a TLR-inhibiting ODN comprises at least one G-tetrad. Alternatively, a TLR-inhibiting ODN comprises one, two, three, four, or more G-tetrads. When a TLR-inhibiting ODN comprises more than one G-tetrad, the G-tetrads can be arranged contiguously. Alternatively, the G-tetrads can be separated by one or more different deoxynucleotides, such as one, two, three, four, five, ten, fifteen, twenty, or more deoxynucleoties. In one embodiment, the G-tetrads are separated by fewer than 20 other deoxynucleotides. Other suitable inhibiting ODNs include the synthetic ODNs having the sequences: TCCTAACGGGGAAGT (SEQ. NO. 3), TCCTGGAGGGGTTGT (SEQ. NO. 4) (see O. Duramad et al., J. Immunol., Vol. 174, 5193 (2005)), TCCTGGCGGGCAAGT (SEQ. NO. 5), TCCTGGCGGGTAAGT (SEQ. NO. 6), TCCTGGCGGGAAAGT (SEQ. NO. 7), TCCTGCAGGGTAAGT (SEQ. NO. 8) (see L. L. Stunz et al., Eur. J. Immunol., Vol. 32, 1212 (2002).

In one embodiment, ODNs comprising one or more G-tetrads can self-assemble into four-stranded helices stabilized by planar Hoogsteen base-paired quartets of guanosine. Such four-stranded ODNs are also within the scope of the present invention.

In another embodiment, a composition of the present invention comprises a TLR-inhibiting ODN that comprises two, three, four, five, or more TTAGGG motifs. In a preferred embodiment, a TLR-inhibiting ODN comprises four TTAGGG motifs. In another embodiment, four TTAGGG motifs are arranged contiguously.

In still another embodiment, a composition of the present invention comprises a TLR-inhibiting ODN that comprises two, three, four, five, or more repeats of any one of SEQ. NO. 2-SEQ. NO. 8, or a combination thereof.

In yet another aspect, a composition of the present invention comprises an effective amount of chloroquine, hydroxychloroquine, quinacrine, 9-aminoacridine, 4-aminoquinoline, or a mixture thereof, for inhibiting the activity of TLR9. These compounds have been shown to block the immunostimulatory action of CpG ODN and induce remission of rheumatoid arthritis (“RA”) and systemic lupus erythematosus (“SLE”). R. N. Bhattacharjee et al., Mini Rev. Med. Chem., Vol. 5, 287 (2006); D. E. Macfarlane et al., J. Immunol., Vol. 160, 1122 (1998). Specifically, chloroquine has been used clinically for the treatment of RA and SLE. Chloroquine blocks TLR9-dependent signaling through inhibition of the pH-dependent maturation of endosomes by acting as a basic substance to neutralize acidification in the vesicles. H. Hacker et al., EMBO J., Vol. 17, 6230 (1998). Therefore, chloroquine can act in a composition of the present invention as a TLR9 immunomodulatory agent.

In a further aspect, a composition of the present invention comprises an inhibitor to an expression of a human TLR. In one embodiment, such an inhibitor comprises a ligand of vitamin D receptor (“VDR”) or a VDR agonist. In another embodiment, such a ligand of VDR or VDR agonist comprises vitamin D or a vitamin-D analogue. A suitable vitamin-D analogue is calcipotriol ((1R,3S)-5-[2-[(1R,3aR,7aS)-1-[(2S)-5-cyclopropyl-5-hydroxy-pent-3-en-2-yl]-7a-methyl-2,3,3a,5,6,7-hexahydro-1H-inden-4-ylidene]ethylidene]-4-methylidene-cyclohexane-1,3-diol). In still another embodiment, such a ligand is vitamin D₂ (ergocalciferol or calciferol) or vitamin D₃ (1,25-dihydroxycholeciciferol or calcitriol). In yet another embodiment, such a ligand is vitamin D₃. It has been accepted that vitamin D₃ is a bona-fide hormone involved in cell growth, differentiation, and immunomodulation. The active form of vitamin D mediates immunological effects by binding to nuclear VDR, which is present in virtually all tissues and cell types, including both innate and acquired immune cells. Y. Y. Yee et al., Mini Rev. Med. Chem., Vol. 5, 761 (2005). Activated VDR can antagonize the action of transcription factors NF-AT and NF-κB. Id. Thus, activated VDR or vitamin D₃ have been shown to inhibit the expression of proinflammatory cytokines, such as IL-2, IL-6, IL-8, IL-12, TNF-α, IFN-γ, and GM-CSF. In addition, vitamin D₃ enhances the production of IL-10 and promotes dendritic cell (“DC”) apoptosis, and, thus, inhibits DC-dependent activation of T cells. E. van Etten et al., J. Steroid Biochem. Mol. Biol., Vol. 97, No. 1-2, 93 (2005). Moreover, there is evidence indicating that vitamin D₃ diminishes the expression of TLR2 and TLR4 in monocytes. K. Sadeghi et al., Eur. J. Immunol., Vol. 36, 361 (2006). Thus, vitamin D₃ or its analogues, or other VDR agonists can reduce the sensitization of these cells to MEMs, such as lipoproteins and lipopeptides of a variety of Gram-negative bacteria, peptidoglycan and lipoteicholic acid of Gram-positive bacteria, lipoarabinomannan of Mycobacteria, and other atypical lipopolysaccharides. Consequently, application of a composition of the present invention containing a vitamin D, a vitamin-D analogue, or a VDR agonist can reduce the risk of development, or the severity, of an inappropriate immune response.

In another aspect, an antagonist to a human TLR, an antagonist to a coreceptor of a human TLR, a compound capable of inhibiting activation of a human TLR signaling pathway (“inhibitor of a TLR”) is included in a composition of the present invention in an amount from about 0.0001 to about 5 percent by weight of the composition. Alternatively, such an antagonist or an inhibitor of a TLR is present in a composition of the present invention in an amount from about 0.001 to about 2 percent (or from about 0.001 to about 1, or from about 0.001 to about 0.5, or from about 0.001 to about 0.2, or from about 0.001 to about 0.1, or from about 0.01 to about 0.1, or from about 0.01 to about 0.5, or from about 0.001 to about 0.01, or from about 0.001 to about 0.1 percent) by weight of the composition.

In still another aspect, a composition of the present invention is a solution used for storing, cleaning, wetting, rinsing, or disinfecting contact lenses.

In yet another aspect, the use of such a solution further provides a contact-lens wearer a benefit of reducing the risk of development, or severity, of inappropriate immune response in the eye.

In a further aspect, the use of such a solution further provides a contact-lens wearer a benefit of reducing the risk of development, or severity, of CLACIs.

### Other Suitable Ingredients in a Composition of the Present Invention

In addition to an antagonist to at least a human TLR, an antagonist to a coreceptor of a human TLR, or an inhibitor to a human TLR or a coreceptor thereof, a composition of the present invention comprises a liquid medium. In one embodiment, the liquid medium comprises an aqueous solution.

In another aspect, a composition of the present invention further comprises a material selected from the group consisting of preservatives, antimicrobial agents, surfactants, buffers, tonicity-modifying agents, chelating agents, viscosity-modifying agents, co-solvents, oils, humectants, emollients, stabilizers, antioxidants and combinations thereof.

Water-soluble preservatives that may be employed in a composition of the present invention include benzalkonium chloride, benzoic acid, benzoyl chloride, benzyl alcohol, chlorobutanol, calcium ascorbate, ethyl alcohol, potassium sulfite, sodium ascorbate, sodium benzoate, sodium bisulfite, sodium bisulfate, sodium thiosulfate, thimerosal, methylparaben, ethylparaben, propylparaben, polyvinyl alcohol, and phenylethyl alcohol. Other preservatives useful in the present invention include, but are not limited to, the FDA-approved preservative systems for food, cosmetics, and pharmaceutical preparations. These agents may be present in individual amounts of from about 0.001 to about 5 percent by weight (preferably, about 0.01 percent to about 2 percent by weight).

In one embodiment, a composition of the present invention comprises an anti-microbial agent. Non-limiting examples of antimicrobial agents include the quaternary ammonium compounds and bisbiguanides. Representative examples of quaternary ammonium compounds include benzalkonium halides and balanced mixtures of n-alkyl dimethyl benzyl ammonium chlorides. Other examples of antimicrobial agents include polymeric quaternary ammonium salts used in ophthalmic applications such as poly[(dimethyliminio)-2-butene-1,4-diyl chloride], [4-tris(2-hydroxyethyl)ammonio]-2-butenyl-w-[tris(2-hydroxyethyl)ammonio]dichloride (chemical registry number 75345-27-6) generally available as Polyquaternium 1® from ONYX Corporation.

Non-limiting examples of antimicrobial biguanides include the bis(biguanides), such as alexidine or chlorhexidine or salts thereof, and polymeric biguanides such as polymeric hexamethylene biguanides (“PHMB”) and their water-soluble salts, which are available, for example, from Zeneca, Wilmington, Del.

In one aspect, a composition of the present invention includes a disinfecting amount of an antimicrobial agent that will at least reduce the microorganism population in the formulations employed. Preferably, a disinfecting amount is that which will reduce the microbial burden by two log orders in four hours and more preferably by one log order in one hour. Most preferably, a disinfecting amount is an amount which will eliminate the microbial burden on a contact lens when used in regimen for the recommended soaking time (FDA Chemical Disinfection Efficacy Test—July, 1985 Contact Lens Solution Draft Guidelines). Typically, such agents are present in concentrations ranging from about 0.00001 to about 0.5 percent (w/v); preferably, from about 0.00003 to about 0.5 percent (w/v); and more preferably, from about 0.0003 to about 0.1 percent (w/v).

In another aspect, a composition of the present invention comprises a surfactant. Suitable surfactants can be amphoteric, cationic, anionic, or non-ionic, which may be present (individually or in combination) in amounts up to 15 percent, preferably up to 5 percent weight by volume (w/v) of the total composition (solution). In one embodiment, the surfactant is an amphoteric or non-ionic surfactant, which when used imparts cleaning and conditioning properties. The surfactant should be soluble in the lens care solution and non-irritating to eye tissues. Many non-ionic surfactants comprise one or more chains or polymeric components having oxyalkylene (—O—R—) repeats units wherein R has 2 to 6 carbon atoms. Preferred non-ionic surfactants comprise block polymers of two or more different kinds of oxyalkylene repeat units. Satisfactory non-ionic surfactants include polyethylene glycol esters of fatty acids, polysorbates, polyoxyethylene, or polyoxypropylene ethers of higher alkanes (C₁₂-C₁₈). Non-limiting examples of the preferred class include polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., p. 291 and the contents of chapter 22, Lippincott Williams & Wilkins, New York, 2006); the contents of these sections are incorporated herein by reference. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1 weight percent).

Various other ionic as well as amphoteric and anionic surfactants suitable for in the invention can be readily ascertained, in view of the foregoing description, from McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C.

Amphoteric surfactants suitable for use in a composition according to the present invention include materials of the type offered commercially under the trade name “Miranol.” Another useful class of amphoteric surfactants is exemplified by cocoamidopropyl betaine, commercially available from various sources.

The foregoing surfactants will generally be present in a total amount from 0.001 to 5 percent weight by volume (w/v), or 0.01 to 5 percent, or 0.01 to 2 percent, or 0.1 to 1.5 percent (w/v).

In another aspect, the pH of a composition of the present invention is maintained within the range of 5 to 8, preferably about 6 to 8, more preferably about 6.5 to 7.8. Non-limiting examples of suitable buffers include boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred, particularly for enhancing the efficacy of biguanides, when they are used in compositions of the present invention. Generally, buffers will be used in amounts ranging from about 0.05 to 2.5 percent by weight, and preferably, from 0.1 to 1.5 percent. In certain embodiments of this invention, the compositions comprise a borate or mixed phosphate buffer, containing one or more of boric acid, sodium borate, potassium tetraborate, potassium metaborate, or mixtures of the same.

In addition to buffering agents, in some instances it may be desirable to include chelating or sequestering agents in the present compositions in order to bind metal ions, which might otherwise react with the lens and/or protein deposits and collect on the lens. Ethylene-diaminetetraacetic acid (“EDTA”) and its salts (disodium) are preferred examples. They are usually added in amounts ranging from about 0.01 to about 0.3 weight percent. Other suitable sequestering agents include phosphonic acids, gluconic acid, citric acid, tartaric acid, and their salts; e.g., sodium salts.

In another aspect, compositions of the present invention comprise a tonicity-adjusting agent, to approximate the osmotic pressure of normal lacrimal fluid, which is equivalent to a 0.9 percent solution of sodium chloride or 2.5 percent of glycerol solution. Examples of suitable tonicity-adjusting agents include, but are not limited to, sodium and potassium chloride, dextrose, glycerin, calcium and magnesium chloride. These agents are typically used individually in amounts ranging from about 0.01 to 2.5 percent (w/v) and preferably, form about 0.2 to about 1.5 percent (w/v). Preferably, the tonicity-adjusting agent will be employed in an amount to provide a final osmotic value of 200 to 450 mOsm/kg, more preferably between about 250 to about 350 mOsm/kg, and most preferably between about 280 to about 320 mOsm/Kg.

In another aspect, it may be desirable to include one or more water-soluble viscosity-modifying agents in the compositions of the present invention. Because of their demulcent effect, viscosity-modifying agents have a tendency to enhance the lens wearer's comfort by means of a film on the lens surface cushioning impact against the eye. Included among the water-soluble viscosity-modifying agents are the cellulose polymers like hydroxyethyl or hydroxypropyl cellulose, carboxymethyl cellulose and the like. Such viscosity-modifying agents may be employed in amounts ranging from about 0.01 to about 4 weight percent or less. The present compositions may also include optional demulcents.

In addition, a composition of the present invention can include additives such as co-solvents, oils, humectants, emollients, stabilizers, or antioxidants for a variety of purposes. These additives may be present in amounts sufficient to provide the desired effects, without impacting the performance of other ingredients.

### Demonstration of Inhibition of Production of Pro-Inflammatory Chemokines

EXPERIMENT 1: Inhibitory ODN suppression of neutrophils activated by synthetic stimulatory ODN sequence, bacterial DNA, and whole bacteria, but not by specific TLR ligand Pam3Cys or LPS.

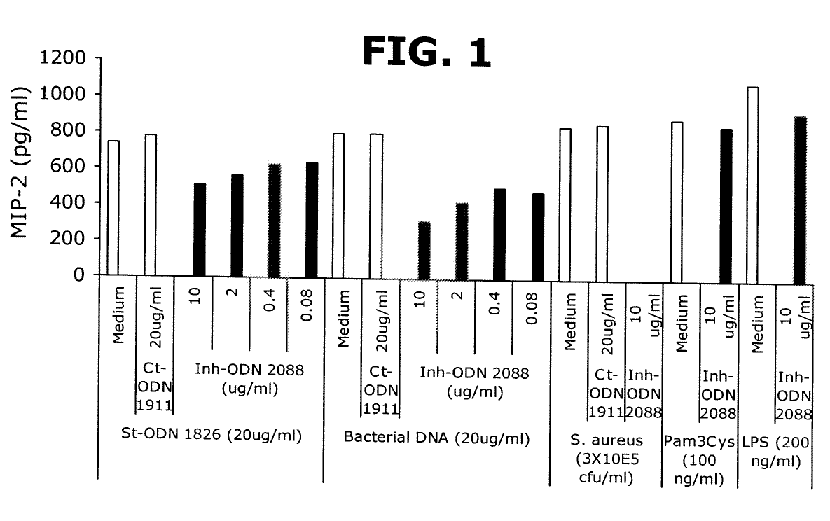

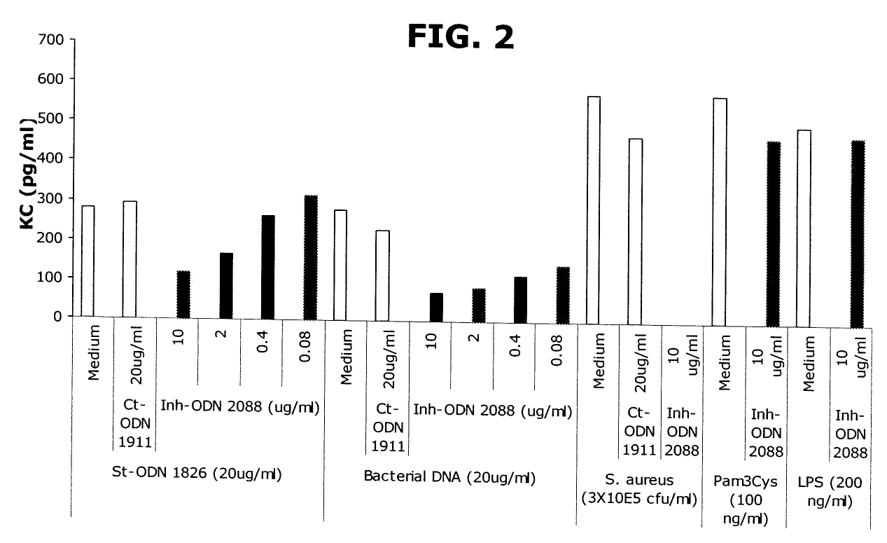

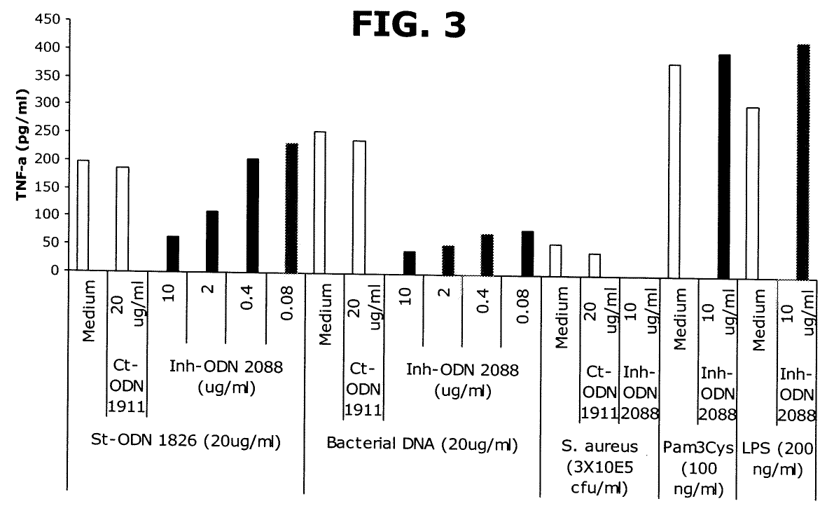

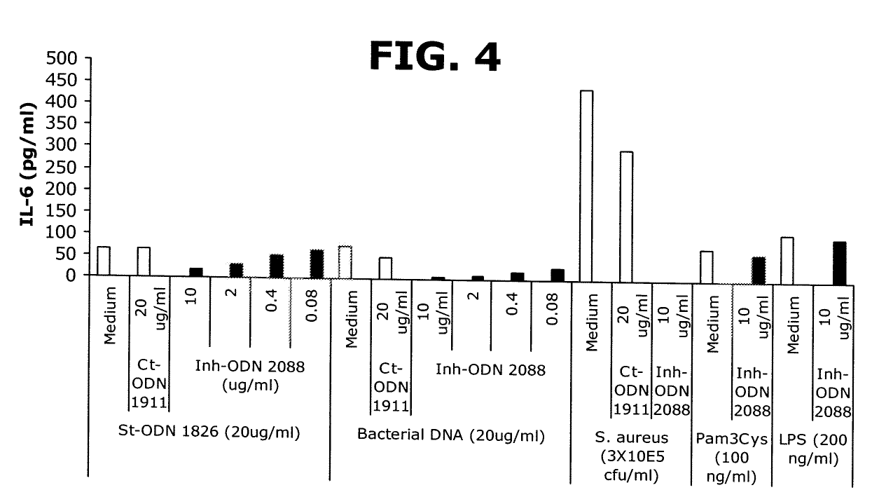

In one experiment, mouse peritoneal neutrophils were isolated from C57BL/6 mice that had received intraperitoneal injection of 1% casein solution containing 0.5 mM MgCl₂ and 0.99 mM CaCl₂ 16 hours and 3 hours prior to harvesting in Hank's balanced salt solution (“HBSS”) lavage. Collected cells were centrifuged (2000 rpm, 10 min) and washed twice in HBSS, prior to separation of granulocytes by Percol gradient at 31,500 rpm for 20 min. Cells were washed twice and resuspended in Dubelco's modified eagle's medium (“DMEM”) containing 10% fetal calf serum (Invitrogen, Basel Switzerland). Purity of 98% neutrophils was verified by Diff-Quik stain (VWR, Bridgeport, N.J.). Neutrophils (1×10⁵/well) were pre-incubated with 100 ng/ml GM-CSF at 37° C. for 1 hour prior to exposure to compositions of the present invention comprising 0.08-10 μg/ml of inhibitory ODN 2088 (InvivoGen, San Diego, Calif.; sequence disclosed above) or a control composition containing 20 μg/ml of the control ODN 1911 (Operon Qiagen, Valencia, Calif.; having a sequence of TCCAGGACTTTCCTCAGGTT), or the medium only, for 30 minutes prior to activation with 20 μg/ml of stimulatory ODN 1826 (Operon Qiagen, Valencia, Calif.; having a sequence of TCCATGACGTTCCTGACGTT); 20 μg/ml of endotoxin-free DNA from E. coli K12 (InvivoGen, San Diego, Calif.); killed Staphylococcus aureus strain E2061740 (3×10⁵ cfu/ml); 100 ng/ml of Pam3Cys (synthetic lipopeptide (S)-(2,3-bis(palmitoyloxy)-(2RS)-propyl)-N-palmitoyl-(R)-Cys-(S)-Ser-(S)-Lys₄-OH, EMC Microcollections, Tubingen, Germany); or 200 ng/ml of LPS (ultra pure lipopolysaccharide from E. coli 0111:B4 strain, InvivoGen, San Diego, Calif.). After 15 hours at 37° C., supernates were collected for ELISA assay (R&D Systems, Minneapolis, Minn.) for pro-inflammatory cytokines macrophage inflammatory protein-2 (“MIP-2”), keratinocyte-derived chemokines (“KC”), IL-6, and TNF-α. Results of cytokine concentrations are shown in FIGS. 1-4. The composition containing the inhibitory ODN 2088 inhibited pro-inflammatory cytokine production by neutrophils upon exposure to the synthetic stimulatory ODN 1826 or bacterial DNA in a dose dependent manner Furthermore, the composition containing the inhibitory ODN 2088 prevented the production of pro-inflammatory cytokines, as exhibited by the nondetectable levels of these four cytokines, when neutrophils were activated with killed Staphylococcus aureus. The production of these pro-inflammatory cytokines was not affected when neutrophils activated by Pam3Cys or LPS were treated with a composition comprising the inhibitory ODN 2088. This not surprising in view of the fact that the inhibitory ODN 2088 inhibits the activation of TLR9 while LPS and Pam3Cys activate TLR4 and TLR2, respectively. Other inhibitors of TLR2 and TLR4 activation should be effective in suppressing corneal infiltrate induced by LPS and Pam3Cys, respectively.

EXPERIMENT 2-1: Inhibitory ODN suppression of mouse keratitis induced by synthetic stimulatory ODN sequence or bacterial DNA, but not by TLR ligand Pam3Cys or LPS.

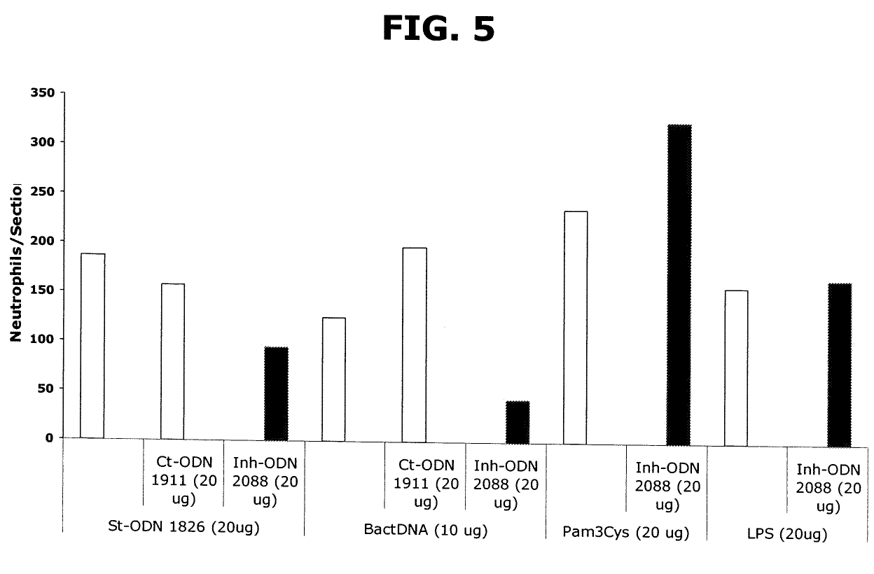

In this experiment, 1 μl of test solution containing 20 μg/ml of the synthetic stimulatory ODN 1826, 10 μg/ml of endotoxin-free DNA from E. coli K12, 20 μg/ml of Pam3Cys, or 20 μg/ml LPS, along with a composition of the present invention containing the inhibitory ODN 2088, the control composition containing 20 μg/ml of ODN 1911, or medium only, was applied to a 1 mm² abraded area of central C57BL/6 mouse cornea that had been marked by sterile trephine (Miltex, Tuttlingen, Germany) and abraded with an Alger brush II (Alger, PagoVista, Texas). After 24 hours, the corneal infiltrate was determined as the number of neutrophils per corneal section. The results are shown in FIG. 5. The inhibitory ODN 2088 reduced the number of infiltrating neutrophils in response to the stimulatory ODN 1826 or bacterial DNA. The inhibitory ODN 2088 was not effective in suppressing corneal infiltrates in response to Pam3Cys or LPS activation because ODN 2088 inhibits TLR 9 activation while LPS and Pame3Cys activate TLR2 and TLR4, respectively. Other inhibitors of TLR2 and TLR4 activation should be effective in suppressing corneal infiltrate induced by Pam3Cys and LPS, respectively.

EXPERIMENT 2-2: Inhibitory ODN suppression of mouse pro-inflammatory cytokines induced by stimulatory ODN.

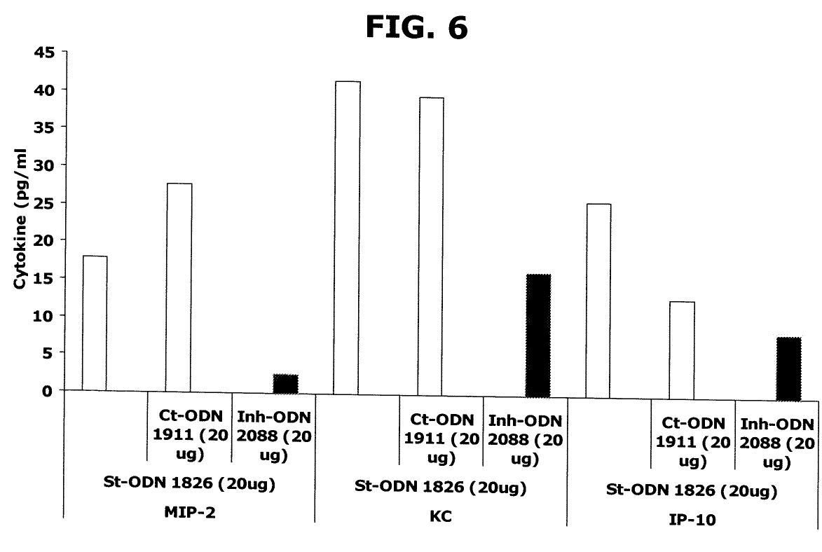

In this experiment, 1 μl of test solution containing 20 μg/ml of the synthetic stimulatory ODN 1826, along with a composition of the present invention containing 20 μg/ml of the inhibitory ODN 2088, or a control composition containing 20 μg/ml of the control ODN 1911, or the medium only, was applied to a 1 mm² abraded area of central C57BL/6 mouse cornea that had been marked by sterile trephine (Miltex, Tuttlingen, Germany) and abraded with an Alger brush II (Alger, PagoVista, TX). After 5 hours, the corneal epithelium was separated after 20 minutes in 20 mM EDTA at 37° C. and placed into RPMI 1640 medium. Samples were disrupted by sonication for 88 seconds with 40% duty cycle (Vibracell; Sonics and Material, Danbury, Conn.). Cytokines were measured by ELISA assay (R&D Systems, Minneapolis, Minn.) for the pro-inflammatory cytokines MIP-2, KC, and human interferon-inducible protein 10 (“IP-10”). The results are shown in FIG. 6. The inhibitory ODN 2088 reduced cytokine response to the stimulatory ODN 1826 for all three cytokines measured.

EXPERIMENT 3: Inhibitory ODN and vitamin D suppression of TLR ligand activation of human cell lines.

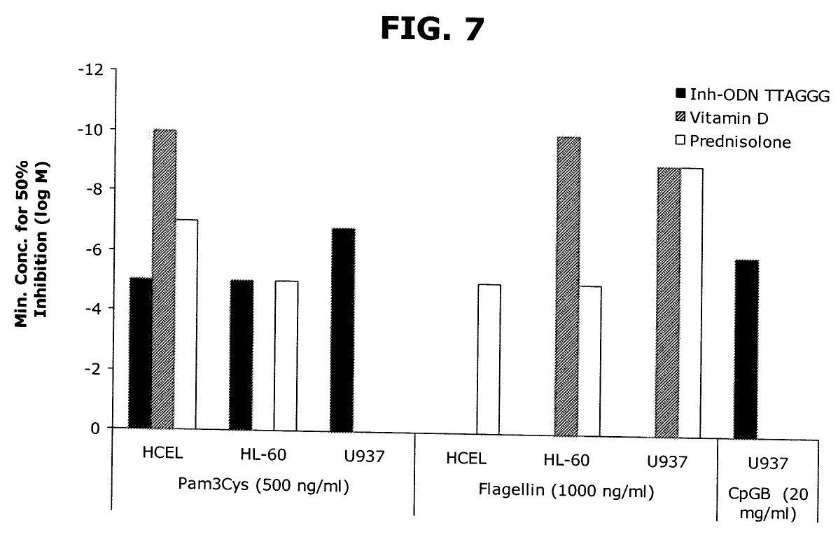

Three human cell lines representative of immune responsive cells of the ocular surface (HCEL, a human corneal epithelial cell line representative of cells present on the ocular surface; HL-60, a neutrophil-like cell line representative of neutrophils present in the tear layer, especially in the closed eye; and U937, a macrophage cell line representative of dendritic cells of the cornea, especially of those at the limbus) were exposed to various concentrations of compositions of the present invention containing the inhibitory ODN TTAGGG (InvivoGen, San Diego, Calif.) and vitamin D (1α,25-Dihydroxyvitamin D₃, Sigma-Aldrich, St. Louis, Mo.) and control compositions containing prednisolone (1,4-Pregnadiene-11β,17α,21-triol-3,20-dione, Sigma-Aldrich, St. Louis, Mo.) for 1 hour prior to activation by the TLR ligand Pam3Cys for 6 hour, flagellin (flagellin purified from Salmonella typhimurium, InvivoGen, San Diego, Calif.) for 24 hr, or the stimulatory CpG type B ODN 2006 (Invivogen, San Diego, Calif.) for 24 hours. After incubation at 37° C., supernates were collected for ELISA assay (R&D Systems, Minneapolis, Minn.) for the pro-inflammatory cytokine CXCL8 (“IL-8”). Results of cytokine concentrations are shown in FIG. 7. Both the inhibitory ODN TTAGGG and vitamin D inhibited cytokine response to TLR ligand activation of each cell line in an inhibitor-specific manner. The inhibitory ODN TTTAGGG reduced the cytokine response of each cell type to Pam3Cys, and of the U937 cell line to the stimulatory CpGB ODN 2006 activation. Vitamin D reduced the cytokine response to Pam3Cys activation of HCEL line and the flagellin activation of HL-60 and U937 lines. Prednisolone inhibited Pam3Cys and flagellin activation of each cell line, except Pam3Cys activation of U937 cell line. Inhibition of the stimulatory ODN CpGB ODN 2006 was only tested with inhibitory ODN TTAGGG on U937 cells.

The following examples serve to illustrate some non-limiting compositions of the present invention.

### EXAMPLE 1

The ingredients shown in Table 1 are mixed to form a contact lens-treating solution that can provide an added benefit of reducing risk of development, or severity, of inappropriate immune response in an eye. In particular, the solution can provide an added benefit of reducing risk of development, or severity, of CLACIs in contact lens wearers.

TABLE 1

| Ingredient | Amount (percent w/v) |
|---|---|
| Polyhexamethylenebiguanide HCl (as a 20 | 0.00047 |
| percent w/w/solution available under the mark | |
| Cosmocil CQ, from ICI Chemical Co.) | |
| Boric acid | 0.64 |
| Sodium borate | 0.12 |
| Edetate disodium | 0.11 |
| Sodium chloride | 0.49 |
| Poloxamine (Tetronic ® 1107 from BASF) | 1.00 |
| Tetrasodium etidronate (as a 30 percent w/w/ | 0.01 |
| solution, available under the mark | |
| DeQuest ® 2016 from Monsanto Co.) | |
| ODN 2088 | 0.005 |
| Hydrochloric acid (1N) or sodium hydroxide | as required to |
| (1N) | adjust pH to7-7.5 |
| Purified water | q.s. to 100 |

### EXAMPLE 2

The ingredients shown in Table 2 are mixed to form a contact lens-treating solution that can provide an added benefit of reducing risk of development, or severity, of inappropriate immune response in an eye. In particular, the solution can provide an added benefit of reducing risk of development, or severity, of CLACIs in contact lens wearers.

TABLE 2

| Ingredient | Amount (percent w/v) |
|---|---|
| Polyhexamethylenebiguanide HCl (as a 20 | 0.00047 |
| percent w/w/ solution available under the mark | |
| Cosmocil CQ, from ICI Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w/ | 0.01 |
| solution, available under the mark | |
| DeQuest ® 2016 from Monsanto Co.) | |
| ODN 2088 | 0.01 |
| Hydrochloric acid (1N) or sodium hydroxide | as required to |
| (1N) | adjust pH to 7-7.5 |
| Purified water | q.s. to 100 |

### EXAMPLE 3

The ingredients shown in Table 3 are mixed to form a contact lens-treating solution that can provide an added benefit of reducing risk of development, or severity, of inappropriate immune response in an eye. In particular, the solution can provide an added benefit of reducing risk of development, or severity, of CLACIs in contact lens wearers.

TABLE 3

| | Amount |
|---|---|
| Ingredient | (percent w/v) |
| Polyhexamethylenebiguanide HCl (as a 20 percent w/w/ | 0.0006 |
| solution available under the mark Cosmocil CQ, from ICI | |
| Chemical Co.) | |
| Phosphate buffer | 0.5 |
| Edetate disodium | 0.11 |
| Sodium chloride | 0.49 |
| Polysorbate 80 | 0.7 |
| Tetrasodium etidronate (as a 30 percent w/w/ solution, | 0.01 |
| available under the mark DeQuest ® 2016 from Monsanto | |
| Co.) | |
| Vitamin D₃ | 0.2 |
| Purified water | q.s. to 100 |

### EXAMPLE 4

The ingredients shown in Table 4 are mixed to form a contact lens-treating solution that can provide an added benefit of reducing risk of development, or severity, of inappropriate immune response in an eye. In particular, the solution can provide an added benefit of reducing risk of development, or severity, of CLACIs in contact lens wearers.

TABLE 4

| Ingredient | Amount (percent w/v) |
|---|---|
| Polyhexamethylenebiguanide HCl (as a 20 | 0.0005 |
| percent w/w/ solution available under the mark | |
| Cosmocil CQ, from ICI Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w/ | 0.01 |
| solution, available under the mark DeQuest ® | |
| 2016 from Monsanto Co.) | |
| Purified anti-human TLR2 antibody (for example | 0.03 |
| from eBioscience, San Diego, California) | |
| Hydrochloric acid (1N) or sodium hydroxide | as required to |
| (1N) | adjust pH to 7-7.5 |
| Purified water | q.s. to 100 |

### EXAMPLE 5

The ingredients shown in Table 5 are mixed to form a contact lens-treating solution that can provide an added benefit of reducing risk of development, or severity, of inappropriate immune response in an eye. In particular, the solution can provide an added benefit of reducing risk of development, or severity, of CLACIs in contact lens wearers.

TABLE 5

| | Amount |
|---|---|
| Ingredient | (percent w/v) |
| Polyhexamethylenebiguanide HCl (as a 20 percent w/w/ | 0.0005 |
| solution available under the mark Cosmocil CQ, from ICI | |
| Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w/ solution, | 0.01 |
| available under the mark DeQuest ® 2016 from Monsanto | |
| Co.) | |
| Soluble TLR4 consisting of the putative extracellular | 0.02 |
| domain (Met¹-Lys⁶³¹, see H. Mitsuzawa et al., J. | |
| Immunol., Vol. 177, 8133 (2006) and N. Hyakushima et | |
| al., J. Immunol., Vol. 173, 6949 (2004)) | |
| Hydrochloric acid (1 N) or sodium hydroxide (1 N) | as required |
| | to adjust pH |
| | to 7-7.5 |
| Purified water | q.s. to 100 |

### EXAMPLE 6

The ingredients shown in Table 6 are mixed to form a contact lens-treating solution that can provide an added benefit of reducing risk of development, or severity, of inappropriate immune response in an eye. In particular, the solution can provide an added benefit of reducing risk of development, or severity, of CLACIs in contact lens wearers.

TABLE 6

| | Amount |
|---|---|
| Ingredient | (percent w/v) |
| Polyhexamethylenebiguanide HCl (as a 20 percent w/w/ | 0.00047 |
| solution available under the mark Cosmocil CQ, from ICI | |
| Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w/ solution, | 0.01 |
| available under the mark DeQuest ® 2016 from | |
| Monsanto Co.) | |
| Chloroquine | 0.001 |
| ODN 2088 | 0.01 |
| Hydrochloric acid (1N) or sodium hydroxide (1N) | as required to |
| | adjust pH to |
| | 7-7.5 |
| Purified water | q.s. to 100 |

### EXAMPLE 7

The ingredients shown in Table 7 are mixed to form a contact lens-treating solution that can provide an added benefit of reducing risk of development, or severity, of inappropriate immune response in an eye. In particular, the solution can provide an added benefit of reducing risk of development, or severity, of CLACIs in contact lens wearers.

TABLE 7

| | Amount |
|---|---|
| Ingredient | (percent w/v) |
| Polyhexamethylenebiguanide HCl (as a 20 percent w/w/ | 0.0005 |
| solution available under the mark Cosmocil CQ, from ICI | |
| Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w/ solution, | 0.01 |
| available under the mark DeQuest ® 2016 from | |
| Monsanto Co.) | |
| Recombinant soluble MD-2 (see H. Mitsuzawa et al., J. | 0.02 |
| Immunol., Vol. 177, 8133 (2006) and N. Hyakushima et | |
| al., J. Immunol., Vol. 173, 6949 (2004)) | |
| Hydrochloric acid (1N) or sodium hydroxide (1N) | as required to |
| | adjust pH to |
| | 7-7.5 |
| Purified water | q.s. to 100 |

### EXAMPLE 8

The ingredients shown in Table 8 are mixed to form a contact lens-treating solution that can provide an added benefit of reducing risk of development, or severity, of inappropriate immune response in an eye. In particular, the solution can provide an added benefit of reducing risk of development, or severity, of CLACIs in contact lens wearers.

TABLE 8

| | Amount |
|---|---|
| Ingredient | (percent w/v) |
| Polyhexamethylenebiguanide HCl (as a 20 percent w/w/ | 0.0005 |
| solution available under the mark Cosmocil CQ, from ICI | |
| Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w/ solution, | 0.01 |
| available under the mark DeQuest ® 2016 from | |
| Monsanto Co.) | |
| Purified anti-human TLR2 antibody (for example from | 0.01 |
| eBioscience, San Diego, California) | |
| Purified anti-human TLR3 antibody (for example from | 0.01 |
| eBioscience, San Diego, California) | |
| Purified anti-human TLR4 antibody (for example from | 0.01 |
| eBioscience, San Diego, California) | |
| Hydrochloric acid (1N) or sodium hydroxide (1N) | as required to |
| | adjust pH to |
| | 7-7.5 |
| Purified water | q.s. to 100 |

### EXAMPLE 9

The ingredients shown in Table 9 are mixed to form a contact lens-storing solution that can provide an added benefit of reducing risk of development, or severity, of inappropriate immune response in an eye. In particular, the solution can provide an added benefit of reducing risk of development, or severity, of CLACIs in contact lens wearers.

TABLE 9

| | Amount |
|---|---|
| Ingredient | (percent w/v) |
| Polyhexamethylenebiguanide HCl (as a 20 percent w/w/ | 0.0005 |
| solution available under the mark Cosmocil CQ, from ICI | |
| Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w/ solution, | 0.01 |
| available under the mark DeQuest ® 2016 from | |
| Monsanto Co.) | |
| Purified anti-human TLR2 antibody (for example from | 0.01 |
| eBioscience, San Diego, California) | |
| Purified anti-human TLR3 antibody (for example from | 0.01 |
| eBioscience, San Diego, California) | |
| Purified anti-human TLR4 antibody (for example from | 0.01 |
| eBioscience, San Diego, California) | |
| ODN 2088 | 0.01 |
| Hydrochloric acid (1N) or sodium hydroxide (1N) | as required to |
| | adjust pH to |
| | 7-7.5 |
| Purified water | q.s. to 100 |

In another aspect, a preservative other than polyhexamethylenebiguanide HCl may be used in any one of the foregoing formulation, in a suitabley effective amount.

In still another aspect, a composition can be free of preservative if it is formulated to be used as a unit-dose composition. In such a case, the composition is packaged in individual container that is opened and the contents of the container are used only once.

In yet another aspect, a composition of the present invention is used as a contact lens-treating, -storing, -cleaning, -disinfecting, or -wetting solution that can provide an added benefit of reducing risk of development, or severity, of inappropriate immune response in an eye. In particular, the solution can provide an added benefit of reducing risk of development, or severity, of CLACIs in contact lens wearers. For example, a daily-wear or extended-wear contact lens can be contacted, soaked, or stored in a composition of the present invention before being installed in the eye of a wearer. An antagonist to at least one human TLR, an antagonist to a coreceptor of a human TLR, or a compound that is capable of inhibiting an activation of a human TLR signaling pathway, in the composition is transferred on the contact lens to the corneal environment and provides a reduction in the risk of development, or severity, of inappropriate immune response in an eye; in particular, the risk of development, or severity, of CLACIs in the contact lens wearer. Thus, the present invention also provides a method for reducing risk of development, or severity, of an inappropriate immune response in an eye or of CLACIs. The method comprises contacting a contact lens with a composition comprising an antagonist to at least one human TLR, an antagonist to a coreceptor of a human TLR, or a compound that is capable of inhibiting an activation of a human TLR signaling pathway, before placing the contact lens in the eye.

In another aspect, a composition of the present invention is formulated as an eye drop, which is applied in the corneal environment of a contact lens wearer on a periodic basis (for example, daily, once every other day, weekly, bimonthly, or monthly) to provide a reduction in the risk of development, or severity, of inappropriate immune response in an eye; in particular, the risk of development, or severity, of CLACIs in the contact lens wearer. Thus, the present invention also provides a method for reducing risk of development, or severity, of an inappropriate immune response in an eye. The method comprises applying a composition to the eye, wherein the composition comprises an antagonist to at least one human TLR, an antagonist to a coreceptor of a human TLR, or a compound that is capable of inhibiting an activation of a human TLR signaling pathway.

In another aspect, the concentration of an antagonist to at least one human TLR, an antagonist to a coreceptor of a human TLR, or a compound that is capable of inhibiting an activation of a human TLR signaling pathway in a composition of the present invention is in any one of the ranges disclosed herein.

In still another aspect, the present invention provides a method for preparing a composition that can reduce the risk of development, or severity, of inappropriate immune response in an eye; in particular, the risk of development, or severity, of CLACIs in a contact lens wearer. The method comprises combining at least one human TLR, an antagonist to a coreceptor of a human TLR, or a compound that is capable of inhibiting an activation of a human TLR signaling pathway with a pharmaceutically acceptable carrier, diluent, excipient, additive, or combination thereof.

What is claimed is:

1. A composition comprising an antagonist to at least a human TLR, an antagonist to at least a coreceptor of human TLR, a compound that is capable of inhibiting an activation of a human TLR signaling pathway, or a combination thereof; wherein said antagonist, compound, or combination thereof is present at a concentration such that the composition is capable of reducing a risk of development, or severity, of an inappropriate immune response in an eye.

2. The composition of claim 1, wherein the inappropriate immune response in an eye comprises contact lens-associated corneal infiltrates (“CLACIs”).

3. The composition of claim 2, wherein the composition comprises a contact lens-treating, -storing, -cleaning, -disinfecting, or -wetting solution.

4. The composition of claim 1, wherein said at least a human TLR is selected from the group consisting of TLR1, TLR2, TLR3, TLR4, TLR5, TLR6, TLR7, TLR8, TLR9, TLR10, and combinations thereof.

5. The composition of claim 1, wherein said at least a coreceptor of human TLR comprises CD14, MD-2, a combination thereof, or a mixture thereof.

6. The composition of claim 1, wherein said antagonist or said compound is selected from the group consisting of anti-human antibodies of TLR1, TLR2, TLR3, TLR4, TLR5, TLR6, TLR7, TLR8, TLR9, TLR10, CD14, or MD-2; and combinations thereof.

7. The composition of claim 1, wherein said compound that is capable of inhibiting an activation of a human TLR signaling pathway comprises a soluble form of an extracellular domain of a human TLR that recognizes a microbe-expressed molecular structure.

8. The composition of claim 1, wherein said compound that is capable of inhibiting an activation of a human TLR signaling pathway comprises at least a soluble form of CD14 or MD-2.

9. The composition of claim 1, wherein said antagonist or compound comprises a nucleotide sequence selected from the group consisting of SEQ. NO. 1-SEQ. NO. 8, and combinations thereof.

10. The composition of claim 1, wherein said antagonist or compound comprises a nucleotide sequences comprising multiple repeats of any one of SEQ. NO. 1-SEQ. NO. 8.

11. The composition of claim 10, wherein said nucleotide sequence comprises two, three, four, or five repeats of any one of SEQ. NO. 1-SEQ. NO. 8.

12. The composition of claim 1, wherein said antagonist or compound comprises a material selected from the group consisting of chloroquine, hydroxychloroquine, quinacrine, 9-aminoacridine, 4-aminoquinoline, and a mixture thereof.

13. The composition of claim 1, wherein said antagonist or compound comprises a ligand of vitamin D receptor.

14. The composition of claim 13, wherein said ligand of vitamin D receptor comprises vitamin D or an analogue thereof.

15. The composition of claim 13, wherein said ligand of vitamin D receptor comprises vitamin D₂, vitamin D₃, or a mixture thereof.

16. The composition of claim 1, wherein said antagonist or said compound is present in an amount in a range from about 0.0001 to about 5 percent by weight of said composition.

17. The composition of claim 6, wherein said antagonist or said compound is present in an amount in a range from about 0.001 to about 2 percent by weight of said composition.

18. The composition of claim 16, further comprising a material selected from the group consisting of carriers, preservatives, antimicrobial agents, surfactants, buffers, tonicity-modifying agents, chelating agents, viscosity-modifying agents, co-solvents, oils, humectants, emollients, stabilizers, antioxidants, and combinations thereof.

19. The composition of claim 18, wherein the composition has a pH in a range from about 5 to about 8.

20. The composition of claim 18, wherein the composition has a pH in a range from about 6.5 to about 7.8.

21. A composition comprising an antagonist to at least a human TLR, an antagonist to at least a coreceptors of human TLR, a compound that is capable of inhibiting an activation of a human TLR signaling pathway, or a combinations thereof; wherein the composition is capable of reducing a risk of development, or severity, of an inappropriate immune response in an eye; wherein said at least a human TLR comprises TLR1, TLR2, TLR3, TLR4, TLR5, TLR6, TLR7, TLR8, TLR9, TLR10, or a combination thereof; said at least a coreceptors of human TLR comprises CD14, MD-2, a combination thereof, or a mixture thereof; said antagonist or compound is present in an amount from about 0.0001 to about 5 percent by weight of said composition; and said composition has a pH of about 5-8.

22. A method for reducing risk of development, or severity, of an inappropriate immune response in an eye of a subject, the method comprising transferring to an environment of said eye a composition that comprises an antagonist to at least a human TLR, an antagonist to at least a coreceptor of human TLR, a compound that is capable of inhibiting an activation of a human TLR signaling pathway, or a combination thereof.

23. The method of claim 22, wherein said transferring comprising administering a pharmaceutically effective amount of said composition to said eye.

24. The method of claim 23, wherein said amount is effective to reduce the risk of development, or severity, of the inappropriate immune response in the eye.

25. The method of claim 22, wherein said inappropriate immune response comprises CLACIs.

26. The method of claim 25, wherein said transferring comprising contacting a contact lens to be worn by said subject with said composition before installing said contact lens in said subject.

27. The method of claim 26, wherein the composition comprises a contact lens-treating, -storing, -cleaning, -disinfecting, or -wetting solution.

28. The method of claim 25; wherein said at least a human TLR comprises TLR1, TLR2, TLR3, TLR4, TLR5, TLR6, TLR7, TLR8, TLR9, TLR10, or a combination thereof; and said at least a coreceptor of human TLR comprises CD14, MD-2, a combination thereof, or a mixture thereof.

29. The method of claim 25, wherein said antagonist or said compound is selected from the group consisting of anti-human antibodies of TLR1, TLR2, TLR3, TLR4, TLR5, TLR6, TLR7, TLR8, TLR9, TLR10, CD14, or MD-2; and combinations thereof.

30. The method of claim 25, wherein said compound that is capable of inhibiting an activation of a human TLR signaling pathway comprises a soluble form of an extracellular domain of a human TLR that recognizes a microbe-expressed molecular structure, or a soluble form of CD14 or MD-2.

31. The method of claim 25, wherein said antagonist or compound comprises a nucleotide sequence selected from the group consisting of SEQ. NO. 1-SEQ. NO. 8, and combinations thereof.

32. The method of claim 25, wherein said antagonist or compound comprises a nucleotide sequences comprising multiple repeats of any one of SEQ. NO. 1-SEQ. NO. 8.

33. The method of claim 32, wherein said nucleotide sequence comprises two, three, four, or five repeats of any one of SEQ. NO. 1-SEQ. NO. 8.

34. The method of claim 25, wherein said antagonist or compound comprises a material selected from the group consisting of chloroquine, hydroxychloroquine, quinacrine, 9-aminoacridine, 4-aminoquinoline, and a mixture thereof.

35. The method of claim 25, wherein said antagonist or compound comprises a ligand of vitamin D receptor.

36. The method of claim 35, wherein said ligand of vitamin D receptor comprises vitamin D or an analogue thereof.

37. The method of claim 35, wherein said ligand of vitamin D receptor comprises vitamin D₂, vitamin D₃, or a mixture thereof.

38. The method of claim 25, wherein said antagonist or said compound is present in an amount in a range from about 0.0001 to about 5 percent by weight of said composition.

39. A method for reducing a risk of development, or severity, of CLACIs in a subject, the method comprising contacting a contact lens to be worn by the subject with a composition before installing the contact lens in the subject; wherein the composition comprises an antagonist to at least a human TLR, an antagonist to at least a coreceptor of human TLR, a compound that is capable of inhibiting an activation of a human TLR signaling pathway, or a combination thereof; wherein the composition is capable of reducing said risk; and wherein said at least a human TLR comprises TLR1, TLR2, TLR3, TLR4, TLR5, TLR6, TLR7, TLR8, TLR9, TLR10, or a combination thereof; said at least a coreceptor of human TLR comprises CD14, MD-2, a combination thereof, or a mixture thereof; said antagonist or compound is present in an amount from about 0.0001 to about 5 percent by weight of said composition; and said composition has a pH of about 5-8.

40. A method for reducing a risk of development, or severity, of CLACIs in a subject, the method comprising transferring an amount of a composition to an eye of the subject; wherein the composition comprises an antagonist to at least a human TLR, an antagonist to at least a coreceptor of human TLR, a compound that is capable of inhibiting an activation of a human TLR signaling pathway, or a combination thereof; wherein the amount is effective to reduce said risk; and wherein said at least a human TLR comprises TLR1, TLR2, TLR3, TLR4, TLR5, TLR6, TLR7, TLR8, TLR9, TLR10, or a combination thereof, said at least a coreceptor of human TLR comprises CD14, MD-2, a combination thereof, or a mixture thereof; said antagonist or compound is present in an amount from about 0.0001 to about 5 percent by weight of said composition; and said composition has a pH of about 5-8.

41. A method for preparing an ophthalmic composition, the method comprising: (a) combining an antagonist to at least a human TLR, an antagonist to at least a coreceptor of human TLR, a compound that is capable of inhibiting an activation of a human TLR signaling pathway, or a combination thereof with an ophthalmically acceptable carrier to form said ophthalmic composition; wherein said antagonist or said compound is present in said composition in a concentration such that an effective amount can be transferred to a corneal environment of a subject.


SEQUENCE LISTING (9 sequences)

SEQ ID NO: 1 - DNA, 6 bp, Homo sapiens
ttaggg

SEQ ID NO: 2 - DNA, 15 bp, Artificial sequence
  Synthetic oligonucleotide
tcctggcggggaagt

SEQ ID NO: 3 - DNA, 15 bp, Artificial sequence
  Synthetic oligonucleotide
tcctaacggggaagt

SEQ ID NO: 4 - DNA, 15 bp, Artificial sequence
  Synthetic oligonucleotide
tcctggaggggttgt

SEQ ID NO: 5 - DNA, 15 bp, Artificial sequence
  Synthetic oligonucleotide
tcctggcgggcaagt

SEQ ID NO: 6 - DNA, 15 bp, Artificial sequence
  Synthetic oligonucleotide
tcctggcgggtaagt

SEQ ID NO: 7 - DNA, 15 bp, Artificial sequence
  Synthetic oligonucleotide
tcctggcgggaaagt

SEQ ID NO: 8 - DNA, 15 bp, Artificial sequence
  Synthetic oligonucleotide
tcctgcagggtaagt

SEQ ID NO: 9 - protein, 15 aa, Mus musculus
ASPSERTYRILEHIS